(19) United States
(12) Patent Application Publication
(10) Pub. No.: US 2020/0165599 A1
(43) Pub. Date: May 28, 2020

(54) Compositions and Methods for Isolating Target Nucleic Acids

(71) Applicant: Gen-Probe Incorporated, San Diego, CA (US)

(72) Inventor: Ankur Shah, San Diego, CA (US)

(73) Assignee: Gen-Probe Incorporated, San Diego, CA (US)

(21) Appl. No.: 16/611,679

(22) PCT Filed: May 10, 2018
(86) PCT No.: PCT/US2018/032044

§ 371 Date: Nov. 7, 2019

Related U.S. Application Data
(60) Provisional application No. 62/504,900, filed on May 11, 2017.

(51) Int. Cl. C12N 15/10; G01N 1/28

(52) U.S. Cl. CPC C12N 15/1006; G01N 1/28

(57) ABSTRACT

Populations of target capture probes are provided that are useful for nucleic acid separation and purification. The probes of the population comprise a first region that is at least about 12 residues in length and comprises a poly(r) sequence comprising (i) a randomized sequence comprising G and A nucleotides, or (ii) a non-randomized repeating (A and G) sequence; and a second region comprising a first specific binding partner (SBP), wherein the SBP is capable of specifically binding a second specific binding partner (SBP2). Related combinations, methods, uses, kits, and reaction mixtures are also provided.

This application claims the benefit of U.S. Provisional Patent Application No. 62/504,900, filed May 11, 2017, which is incorporated herein by reference for all purposes.

This disclosure relates to the field of molecular biology, more particularly to methods and compositions for nucleic acid isolation from a mixture, such as a sample, by using a population of probes that hybridize to target nucleic acid(s) to allow separation from other components of the mixture.

### SEQUENCE LISTING

The present application is filed with a Sequence Listing in electronic format. The Sequence Listing is provided as a file entitled “2018-05-01_01159-0016-00PCT_Seq_List_ST25.txt” created on May 1, 2018, which is 4,131 bytes in size. The information in the electronic format of the sequence listing is incorporated herein by reference in its entirety.

### I. INTRODUCTION AND SUMMARY

Many molecular biology procedures such as in vitro amplification and in vitro hybridization of nucleic acids include some preparation of nucleic acids to facilitate the subsequent procedure. Methods of nucleic acid purification may isolate all nucleic acids present in a sample, isolate different types of nucleic acids based on physical characteristics, or isolate specific nucleic acids from a sample. Many methods involve complicated procedures, use harsh chemicals or conditions, or require a long time to complete the nucleic acid isolation. Some methods involve use of specialized oligonucleotides, each specific for an intended target nucleic acid which adds complexity to the design, optimization and performance of methods, particularly if isolation of more than one target nucleic acid is desired or if the sequence of the desired target nucleic acid is unknown. Some methods isolate target nucleic acids without requiring a particular target sequence but do not isolate all sequences efficiently. Thus, there remains a need for a simple, efficient, and fast method to separate nucleic acids of interest from other sample components.

Accordingly, the following embodiments are among those provided by the disclosure.

Embodiment 1 is a population of capture probes for isolating a target nucleic acid from a sample, comprising a first region that is at least about 12 residues in length and comprises at least one poly(r) sequence comprising (i) a randomized sequence comprising G and A nucleotides, or (ii) a non-randomized repeating (A and G) sequence; and a second region comprising a first specific binding partner (SBP), wherein the SBP is capable of specifically binding a second specific binding partner (SBP2).

Embodiment 2 is the population of capture probes of embodiment 1, wherein the poly-(r) sequence comprises the randomized sequence comprising G and A nucleotides.

Embodiment 3 is the population of capture probes of embodiment 2, wherein the first region comprises at least about 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides of randomized poly-(r) sequence.

Embodiment 4 is the population of capture probes of any one of the preceding embodiments, wherein the poly-(r) sequence comprises at least about 4, 5, 6, 7, 8, 9, 10, 11, 12, 13, 14, 15, 16, 17, 18, 19, 20, 21, 22, 23, 24, 25, 26, 27, 28, 29, or 30 nucleotides of a non-randomized repeating (A and G) sequence.

Embodiment 5 is the population of capture probes of any one of the preceding embodiments, wherein the first region is at least 13, 14, 15, 16, 17, 18, 19, 20, 21, 22, 23, 24, 25, 26, 27, 28, 29, or 30 nucleotides in length.

Embodiment 6 is the population of capture probes of any one of the preceding embodiments, wherein the first region consists of the randomized G and A nucleotides, the non-randomized repeating (A and G) sequence, or a combination thereof.

Embodiment 7 is the population of capture probes of any one of embodiments 1-5, wherein the first region further comprises a linker sequence between the poly(r) sequence and a second poly(r) sequence, and the second poly(r) sequence comprises (i) a randomized sequence comprising G and A nucleotides, or (ii) a non-randomized repeating (A and G) sequence.

Embodiment 8 is the population of capture probes of embodiment 7, wherein the poly(r) sequence is at least about 6 residues in length and the second poly(r) sequence is at least about 6 residues in length.

Embodiment 9 is the population of capture probes of any one of the preceding embodiments, wherein the first region comprises 2′-O-methyl modified RNA residues.

Embodiment 10 is the population of capture probes of any one of the preceding embodiments, wherein the first region comprises a poly-(r)₁₈, poly-(r)₂₄, or poly-(r)₂₅ sequence.

Embodiment 11 is the population of capture probes of any one of the preceding embodiments, wherein the SBP is a non-nucleic acid moiety.

Embodiment 12 is the population of capture probes of any one of embodiments 1 to 10, wherein the SBP comprises a homopolymeric sequence.

Embodiment 13 is the population of capture probes of embodiment 12, wherein the SBP comprises a dT₃dA₃₀ (SEQ ID NO: 10) or dA₃₀ (SEQ ID NO: 11) sequence.

Embodiment 14 is the population of capture probes of any one of the preceding embodiments, wherein the SBP is situated 3′ to the first region.

Embodiment 15 is a combination comprising the population of capture probes of any one of the preceding embodiments and a second population of capture probes comprising a first region that is at least about 12 residues in length and comprises a poly-(k) sequence comprising (i) a randomized sequence comprising G and U/T nucleotides, or (ii) a non-randomized repeating (G and U/T) sequence; and a second region comprising a third specific binding partner (SBP3), wherein the SBP3 is capable of specifically binding a fourth specific binding partner (SBP4).

Embodiment 16 is the combination of embodiment 15, wherein the SBP and the SBP3 are capable of binding the same SBP2/SBP4.

Embodiment 17 is the combination of embodiment 16, wherein the SBP and the SBP3 are identical to each other.

Embodiment 18 is the combination of any one of embodiments 15 to 17, wherein the first region of the second population is at least 13, 14, 15, 16, 17, 18, 19, 20, 21, 22, 23, 24, 25, 26, 27, 28, 29, or 30 nucleotides in length.

Embodiment 19 is the combination of any one of embodiments 15 to 18, wherein the first region of the second population comprises a poly-(k)₁₈, poly-(k)₂₄, or poly-(k)₂₅ sequence.

Embodiment 20 is the combination of any one of embodiments 15 to 19, wherein the first region of the second population consists of randomized G and U/T nucleotides or non-randomized repeating (G and U/T).

Embodiment 21 is a kit or reaction mixture for isolating a target nucleic acid from a sample, said reaction mixture comprising:

Embodiment 22 is a population of capture probes of any of embodiments 1 to 14 or combination of any one of embodiments 15 to 20; and

Embodiment 23 is an SBP2 immobilized on a support.

Embodiment 24 is the kit or reaction mixture of embodiment 21, wherein the SBP and SBP2 are substantially complementary nucleic acid sequences.

Embodiment 25 is the kit or reaction mixture of embodiment 21, wherein the SBP and SBP2 are non-nucleic acid moieties.

Embodiment 26 is the kit or reaction mixture of any one of embodiments 21-23, further comprising a detergent.

Embodiment 27 is the kit or reaction mixture of any one of embodiments 21-24, further comprising lithium or sodium lauryl sulfate and/or lithium hydroxide.

Embodiment 28 is the kit or reaction mixture of any one of embodiments 21-25, comprising the combination of capture probes of any one of embodiments 15-20.

Embodiment 29 is the kit or reaction mixture of embodiment 26, wherein the SBP and the SBP3 are capable of binding the SBP2.

Embodiment 30 is the kit or reaction mixture of embodiment 26, further comprising an SBP4 immobilized on a support.

Embodiment 31 is the kit or reaction mixture of any one of embodiments 21-28, further comprising a solution phase.

Embodiment 32 is the reaction mixture of embodiment 29, wherein the reaction mixture comprises a target nucleic acid in the solution phase and/or associated with the capture probes.

Embodiment 33 is the reaction mixture of embodiment 30, wherein the target nucleic acid is derived from cells that have been treated to release intracellular components into the solution phase.

Embodiment 34 is the reaction mixture of any one of embodiments 29-31, wherein the solution phase comprises a sample from an animal, environmental, food, or industrial source.

Embodiment 35 is the reaction mixture of any one of embodiments 29-32, wherein the solution phase comprises a sample comprising peripheral blood, serum, plasma, cerebrospinal fluid, sputum, or a swab specimen.

Embodiment 36 is a method for isolating a target nucleic acid from a sample, the method comprising: contacting a population of capture probes of any one of embodiments 1 to 14 or combination of any one of embodiments 15 to 20 with a solution containing nucleic acids to form a reaction mixture, wherein the reaction mixture further comprises a support comprising the SBP2; incubating the reaction mixture in conditions that allow hybridization of the first region with the target nucleic acid and that allow for association of the SBP with the SBP2 immobilized to the support, thereby forming a hybridization complex in contact with a solution phase; and separating the support from the solution phase, thereby isolating the target nucleic acid from other components in the sample.

Embodiment 37 is a method for isolating a target nucleic acid from a sample, the method comprising: incubating the reaction mixture of any one of embodiments 21-33 with the sample in conditions that allow hybridization of the first region with the target nucleic acid and that allow for association of the SBP with the SBP2 immobilized to the support, thereby forming a hybridization complex in contact with a solution phase; and separating the support from the solution phase, thereby isolating the target nucleic acid from other components in the sample.

Embodiment 38 is the method of embodiment 34 or 35, wherein the sample contains cells and is treated before the contacting step to release intracellular components into the solution.

Embodiment 39 is the method of embodiment 36, wherein the treatment comprises treating the sample with a solution containing a detergent.

Embodiment 40 is the method of any one of embodiments 34-37, wherein the sample is from an animal, environmental, food, or industrial source.

Embodiment 41 is the method of any one of embodiments 34-38, wherein the sample comprises peripheral blood, serum, plasma, cerebrospinal fluid, sputum, or a swab specimen.

Embodiment 42 is the method of any one of embodiments 34-39, wherein the sample comprises a cellular lysate.

Embodiment 43 is the method of any one of embodiments 34-40, wherein the SBP and SBP2 are non-nucleic acid moieties.

Embodiment 44 is the method of any one of embodiments 34-40, wherein the SBP and SBP2 are substantially complementary nucleic acid sequences.

Embodiment 45 is the method of any one of embodiments 34-42, wherein the combination as recited in any one of embodiments 15-20 is contacted with the solution containing nucleic acids.

Embodiment 46 is the method of embodiment 43, wherein the SBP and the SBP3 are capable of binding the SBP2.

Embodiment 47 is the method of embodiment 43, wherein the reaction mixture further comprises a support comprising the SBP4.

Embodiment 48 is the population, combination, reaction mixture, or method of any one of the preceding embodiments, wherein the target nucleic acid comprises DNA.

Embodiment 49 is the population, combination, reaction mixture, or method of any one of the preceding embodiments, wherein the target nucleic acid comprises RNA.

Embodiment 50 is the population, combination, reaction mixture, or method of any one of the preceding embodiments, wherein the target nucleic acid comprises viral nucleic acid.

Embodiment 51 is the population, combination, reaction mixture, or method of any one of the preceding embodiments, wherein the target nucleic acid comprises prokaryotic nucleic acid.

Embodiment 52 is the population, combination, reaction mixture, or method of any one of the preceding embodiments, wherein the target nucleic acid comprises eukaryotic nucleic acid.

Embodiment 53 is the population, combination, reaction mixture, or method of any one of the preceding embodiments, wherein the target nucleic acid comprises synthetic nucleic acid.

Embodiment 54 is the population, combination, reaction mixture, or method of any one of the preceding embodiments, wherein the target nucleic acid comprises a combination of DNA, RNA, viral nucleic acid, bacterial nucleic acid, eukaryotic nucleic acid, and/or synthetic nucleic acid.

### BRIEF DESCRIPTION OF THE DRAWINGS

FIG. 1 shows ΔCT of 49 clinical specimens comparing extraction using an (r)₁₈/(k)₁₈ capture probe blend vs. using a (k)₁₈ capture probe only as described in Example 2. Each bar represents results from an individual specimen.

### DETAILED DESCRIPTION

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “an oligomer” includes a plurality of oligomers and the like. The conjunction “or” is to be interpreted in the inclusive sense, i.e., as equivalent to “and/or”, unless the inclusive sense would be unreasonable in the context.

It will be appreciated that there is an implied “about” prior to the temperatures, concentrations, times, etc., discussed in the present disclosure, such that slight and insubstantial deviations are within the scope of the present teachings herein. In general, the term “about” indicates insubstantial variation in a quantity of a component of a composition not having any significant effect on the activity or stability of the composition. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

Unless specifically noted, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

### A. Definitions

“Sample” includes any specimen that may contain a target nucleic acid. Samples include “biological samples” which include any tissue or material derived from a living or dead organism that may contain target nucleic acid derived therefrom, including, e.g., peripheral blood, plasma, serum, lymph node, gastrointestinal tissue, cerebrospinal fluid, sputum, a swab specimen, or other body fluids or materials. The biological sample may be treated to physically or mechanically disrupt tissue or cell structure, thus releasing intracellular components into a solution which may further contain enzymes, buffers, salts, detergents and the like, which are used to prepare, using standard methods, a biological sample for analysis. Also, samples may include processed samples, such as those obtained from passing samples over or through a filtering device, or following centrifugation, or by adherence to a medium, matrix, or support.

“Nucleic acid” refers to a multimeric compound comprising two or more covalently bonded nucleosides or nucleoside analogs having nitrogenous heterocyclic bases, or base analogs, where the nucleosides are linked together by phosphodiester bonds or other linkages to form a polynucleotide. Nucleic acids include RNA, DNA, or chimeric DNA-RNA polymers or oligonucleotides, and analogs thereof. A nucleic acid “backbone” may be made up of a variety of linkages, including one or more of sugar-phosphodiester linkages, peptide-nucleic acid bonds (in “peptide nucleic acids” or PNAs, see, e.g., International Patent Application Pub. No. WO 95/32305), phosphorothioate linkages, methylphosphonate linkages, or combinations thereof. Sugar moieties of the nucleic acid may be either ribose or deoxyribose, or similar compounds having known substitutions such as, for example, 2′-methoxy substitutions and 2′-halide substitutions (e.g., 2′-F). Nitrogenous bases may be conventional bases (A, G, C, T, U), analogs thereof (e.g., inosine, 5-methylisocytosine, isoguanine; see, e.g., The Biochemistry of the Nucleic Acids 5-36, Adams et al., ed., 11th ed., 1992; Abraham et al., 2007, BioTechniques 43: 617-24), which include derivatives of purine or pyrimidine bases (e.g., N⁴-methyl deoxygaunosine, deaza- or aza-purines, deaza- or aza-pyrimidines, pyrimidine bases having substituent groups at the 5 or 6 position, purine bases having an altered or replacement substituent at the 2, 6 and/or 8 position, such as 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines, and pyrazolo-compounds, such as unsubstituted or 3-substituted pyrazolo[3,4-d]pyrimidine; U.S. Pat. Nos. 5,378,825, 6,949,367 and International Patent Application Pub. No. WO 93/13121, each incorporated by reference herein). Nucleic acids may include “abasic” residues in which the backbone does not include a nitrogenous base for one or more residues (see. e.g., U.S. Pat. No. 5,585,481, incorporated by reference herein). A nucleic acid may comprise only conventional sugars, bases, and linkages as found in RNA and DNA, or may include conventional components and substitutions (e.g., conventional bases linked by a 2′-methoxy backbone, or a nucleic acid including a mixture of conventional bases and one or more base analogs). Nucleic acids may include “locked nucleic acids” (LNA), in which one or more nucleotide monomers have a bicyclic furanose unit locked in an RNA mimicking sugar conformation, which enhances hybridization affinity toward complementary sequences in single-stranded RNA (ssRNA), single-stranded DNA (ssDNA), or double-stranded DNA (dsDNA) (Vester et al., Biochemistry 43:13233-41, 2004, incorporated by reference herein). Nucleic acids may include modified bases to alter the function or behavior of the nucleic acid, e.g., addition of a 3′-terminal dideoxynucleotide to block additional nucleotides from being added to the nucleic acid. Synthetic methods for making nucleic acids in vitro are well-known in the art although nucleic acids may be purified from natural sources using routine techniques.

The term “polynucleotide” as used herein denotes a nucleic acid chain. Throughout this application, nucleic acids are designated by the 5′-terminus to the 3′-terminus. Synthetic nucleic acids, e.g., DNA, RNA, DNA/RNA chimerics, (including when non-natural nucleotides or analogues are included therein), are typically synthesized “3′-to-5′,” i.e., by the addition of nucleotides to the 5′-terminus of a growing nucleic acid.

A “nucleotide” as used herein is a subunit of a nucleic acid consisting of a phosphate group, a 5-carbon sugar, and a nitrogenous base (also referred to herein as “nucleobase”). The 5-carbon sugar found in RNA is ribose. In DNA, the 5-carbon sugar is 2′-deoxyribose. The term also includes analogs of such subunits, such as a methoxy group at the 2′ position of the ribose (also referred to herein as “2′-O-Me” or “2′-methoxy”). As used herein, unless otherwise indicated, a “T” residue in a 2′-methoxy oligonucleotide is interchangeable with a “U.”

A “non-nucleotide unit” as used herein is a unit that does not significantly participate in hybridization of a polymer. Such units do not, for example, participate in any significant hydrogen bonding with a nucleotide, and would exclude units having as a component one of the five nucleotide bases or analogs thereof.

A “target nucleic acid” as used herein is a nucleic acid comprising a target sequence to be amplified. Target nucleic acids may be DNA or RNA as described herein, and may be either single-stranded or double-stranded. The target nucleic acid may include other sequences besides the target sequence, which may not be amplified.

“Target-hybridizing sequence” is used herein to refer to the portion of an oligomer that is configured to hybridize with a target nucleic acid. Target-hybridizing sequences may but do not necessarily include a linker (e.g., linker sequences or non-nucleotide chains) between segments that hybridize to a target.

The term “region,” as used herein, refers to a portion of a nucleic acid wherein said portion is smaller than the entire nucleic acid. For example, when the nucleic acid in reference is a capture probe, the term “region” may be used to refer to the smaller target-hybridizing portion of the entire oligonucleotide, or the smaller portion that serves as a specific binding partner.

The interchangeable terms “oligomer,” “oligo,” and “oligonucleotide” refer to a nucleic acid having generally less than 1,000 nucleotide (nt) residues, including polymers in a range having a lower limit of about 5 nt residues and an upper limit of about 500 to 900 nt residues. In some embodiments, oligonucleotides are in a size range having a lower limit of about 12 to 15 nt and an upper limit of about 50 to 600 nt, and other embodiments are in a range having a lower limit of about 15 to 20 nt and an upper limit of about 22 to 100 nt. Oligonucleotides may be purified from naturally occurring sources or may be synthesized using any of a variety of well-known enzymatic or chemical methods. The term oligonucleotide does not denote any particular function to the reagent; rather, it is used generically to cover all such reagents described herein. An oligonucleotide may serve various different functions. For example, it may function as a primer if it is specific for and capable of hybridizing to a complementary strand and can further be extended in the presence of a nucleic acid polymerase; it may function as a primer and provide a promoter if it contains a sequence recognized by an RNA polymerase and allows for transcription (e.g., a T7 Primer); and it may function to detect a target nucleic acid if it is capable of hybridizing to the target nucleic acid, or an amplicon thereof, and further provides a detectible moiety (e.g., a fluorophore).

“Amplification” refers to any known procedure for obtaining multiple copies of a target nucleic acid sequence or its complement or fragments thereof. The multiple copies may be referred to as amplicons or amplification products, which can be double-stranded or single-stranded and can include DNA, RNA, or both. Amplification of “fragments” refers to production of an amplified nucleic acid that contains less than the complete target nucleic acid or its complement, e.g., produced by using an amplification oligonucleotide that hybridizes to, and initiates polymerization from, an internal position of the target nucleic acid. Known amplification methods include, for example, replicase-mediated amplification, polymerase chain reaction (PCR), ligase chain reaction (LCR), strand-displacement amplification (SDA), and transcription-mediated or transcription-associated amplification. Replicase-mediated amplification uses self-replicating RNA molecules, and a replicase such as QB-replicase (see. e.g., U.S. Pat. No. 4,786,600, incorporated by reference herein). PCR amplification uses a DNA polymerase, pairs of primers, and thermal cycling to synthesize multiple copies of two complementary strands of dsDNA or from a cDNA (see. e.g., U.S. Pat. Nos. 4,683,195; 4,683,202; and 4,800,159; each incorporated by reference herein). LCR amplification uses four or more different oligonucleotides to amplify a target and its complementary strand by using multiple cycles of hybridization, ligation, and denaturation (see. e.g., U.S. Pat. Nos. 5,427,930 and 5,516,663, each incorporated by reference herein). SDA uses a primer that contains a recognition site for a restriction endonuclease and an endonuclease that nicks one strand of a hemimodified DNA duplex that includes the target sequence, whereby amplification occurs in a series of primer extension and strand displacement steps (see. e.g., U.S. Pat. Nos. 5,422,252; 5,547,861; and 5,648,211; each incorporated by reference herein). Amplification may be linear or exponential.

“Detection probe,” “detection oligonucleotide,” “probe oligomer,” and “detection probe oligomer” are used interchangeably to refer to a nucleic acid oligomer that hybridizes specifically to a target sequence in a nucleic acid, or in an amplified nucleic acid, under conditions that promote hybridization to allow detection of the target sequence or amplified nucleic acid. Detection may either be direct (e.g., a probe hybridized directly to its target sequence) or indirect (e.g., a probe linked to its target via an intermediate molecular structure). Detection probes may be DNA, RNA, analogs thereof or combinations thereof (e.g., DNA/RNA chimerics) and they may be labeled or unlabeled. Detection probes may further include alternative backbone linkages such as, e.g., 2′-O-methyl linkages. A detection probe's “target sequence” generally refers to a smaller nucleic acid sequence region within a larger nucleic acid sequence that hybridizes specifically to at least a portion of a probe oligomer by standard base pairing. A detection probe may comprise target-specific sequences and other sequences that contribute to the three-dimensional conformation of the probe (see. e.g., U.S. Pat. Nos. 5,118,801; 5,312,728; 6,849,412; 6,835,542; 6,534,274; and 6,361,945; and US Patent Application Pub. No. 20060068417; each incorporated by reference herein).

By “stable” or “stable for detection” is meant that the temperature of a reaction mixture is at least 2° C. below the melting temperature of a nucleic acid duplex.

As used herein, a “label” refers to a moiety or compound joined directly or indirectly to a probe that is detected or leads to a detectable signal. Direct labeling can occur through bonds or interactions that link the label to the probe, including covalent bonds or non-covalent interactions, e.g., hydrogen bonds, hydrophobic and ionic interactions, or formation of chelates or coordination complexes. Indirect labeling can occur through use of a bridging moiety or “linker” such as a binding pair member, an antibody or additional oligomer, which is either directly or indirectly labeled, and which may amplify the detectable signal. Labels include any detectable moiety, such as a radionuclide, ligand (e.g., biotin, avidin), enzyme or enzyme substrate, reactive group, or chromophore (e.g., dye, particle, or bead that imparts detectable color), luminescent compound (e.g., bioluminescent, phosphorescent, or chemiluminescent labels), or fluorophore. Labels may be detectable in a homogeneous assay in which bound labeled probe in a mixture exhibits a detectable change different from that of an unbound labeled probe, e.g., instability or differential degradation properties.

“Capture probe,” “capture oligonucleotide,” “capture oligomer,” “target capture oligomer,” and “capture probe oligomer” are used interchangeably to refer to a nucleic acid oligomer that specifically hybridizes to a target sequence in a target nucleic acid by standard base pairing and joins to a binding partner on an immobilized probe to capture the target nucleic acid to a support. One example of a capture oligomer includes two binding regions: a sequence-binding region (e.g., target-specific portion) and an immobilized probe-binding region, usually on the same oligomer, although the two regions may be present on two different oligomers joined together by one or more linkers. Another embodiment of a capture oligomer uses a target-sequence binding region that includes random or non-random poly-GU, poly-GT, or poly U sequences to bind non-specifically to a target nucleic acid and link it to an immobilized probe on a support.

As used herein, an “immobilized oligonucleotide,” “immobilized probe,” “immobilized binding partner,” “immobilized oligomer,” or “immobilized nucleic acid” refers to a nucleic acid binding partner that joins a capture oligomer to a support, directly or indirectly. An immobilized probe joined to a support facilitates separation of a capture probe bound target from unbound material in a sample. One embodiment of an immobilized probe is an oligomer joined to a support that facilitates separation of bound target sequence from unbound material in a sample. Supports may include known materials, such as matrices and particles free in solution, which may be made of nitrocellulose, nylon, glass, polyacrylate, mixed polymers, polystyrene, silane, polypropylene, metal, or other compositions, of which one embodiment is magnetically attractable particles. Supports may be monodisperse magnetic spheres (e.g., uniform size+5%), to which an immobilized probe is joined directly (via covalent linkage, chelation, or ionic interaction), or indirectly (via one or more linkers), where the linkage or interaction between the probe and support is stable during hybridization conditions.

By “complementary” is meant that the nucleotide sequences of similar regions of two single-stranded nucleic acids, or two different regions of the same single-stranded nucleic acid, have a nucleotide base composition that allow the single-stranded regions to hybridize together in a stable double-stranded hydrogen-bonded region under stringent hybridization or amplification conditions. Sequences that hybridize to each other may be completely complementary or partially complementary to the intended target sequence by standard nucleic acid base pairing (e.g., G:C, A:T, or A:U pairing). By “sufficiently complementary” is meant a contiguous sequence that is capable of hybridizing to another sequence by hydrogen bonding between a series of complementary bases, which may be complementary at each position in the sequence by standard base pairing or may contain one or more residues, including abasic residues, that are not complementary. Sufficiently complementary contiguous sequences typically are at least 80%, or at least 90%, complementary to a sequence to which an oligomer is intended to specifically hybridize. Sequences that are “sufficiently complementary” allow stable hybridization of a nucleic acid oligomer with its target sequence under appropriate hybridization conditions, even if the sequences are not completely complementary. When a contiguous sequence of nucleotides of one single-stranded region is able to form a series of “canonical” or “Watson-Crick” hydrogen-bonded base pairs with an analogous sequence of nucleotides of the other single-stranded region, such that A is paired with U or T and C is paired with G, the nucleotides sequences are “completely” complementary (see. e.g., Sambrook et al., Molecular Cloning. A Laboratory Manual, 2nd ed. (Cold Spring Harbor Laboratory Press, Cold Spring Harbor, N.Y., 1989) at §§ 1.90-1.91, 7.37-7.57, 9.47-9.51 and 11.47-11.57, particularly §§ 9.50-9.51, 11.12-11.13, 11.45-11.47 and 11.55-11.57, incorporated by reference herein). It is understood that ranges for percent identity are inclusive of all whole and partial numbers (e.g., at least 90% includes 90, 91, 93.5, 97.687, etc.). Reference to “the complement” of a particular sequence generally indicates a completely complementary sequence unless the context indicates otherwise. Appropriate hybridization conditions are well-known in the art, may be predicted based on sequence composition, or can be determined by using routine testing methods (see. e.g., Sambrook et al., Molecular Cloning. A Laboratory Manual, 2nd ed. (Cold Spring Harbor Laboratory Press, Cold Spring Harbor, N.Y., 1989) at §§ 1.90-1.91, 7.37-7.57, 9.47-9.51 and 11.47-11.57, particularly §§ 9.50-9.51, 11.12-11.13, 11.45-11.47 and 11.55-11.57, incorporated by reference herein).

“Wobble” base pairs refer to a pairing of a G to either a U or a T.

By “nucleic acid hybrid,” “hybrid,” or “duplex” is meant a nucleic acid structure containing a double-stranded, hydrogen-bonded region wherein the region is sufficiently stable to permit separation or purification of the duplex under appropriate conditions. Such hybrids may comprise RNA:RNA, RNA:DNA, or DNA:DNA duplex molecules, among others.

“Separating” or “purifying” means that one or more components of a sample are removed or separated from other sample components. Sample components include target nucleic acids usually in a generally aqueous solution phase, which may also include cellular fragments, proteins, carbohydrates, lipids, and other nucleic acids. “Separating” or “purifying” does not connote any degree of purification. Typically, separating or purifying removes at least 70%, or at least 80%, or at least 95% of the target nucleic acid from other sample components.

References, particularly in the claims, to “the sequence of SEQ ID NO: X” refer to the base sequence of the corresponding sequence listing entry and do not require identity of the backbone (including but not limited to RNA, 2′-O-Me RNA, DNA, or LNA) unless otherwise indicated. Furthermore, T and U residues are to be considered interchangeable for purposes of sequence listing entries unless otherwise indicated, e.g., a subject sequence is considered identical to a SEQ ID NO with a T as the sixth nucleotide regardless of whether the residue at the sixth position in the subject sequence is a T or a U.

### B. Populations of Capture Probes, Methods, and Uses

Provided herein are populations of capture probes for isolating a target nucleic acid from a sample, comprising a first region that is at least about 12 residues in length and comprises at least one poly(r) sequence comprising (i) a randomized sequence comprising G and A nucleotides, or (ii) a non-randomized repeating (A and G) sequence; and a second region comprising a first specific binding partner (SBP), wherein the SBP is capable of specifically binding a second specific binding partner (SBP2). “Poly-(r)” is used as an abbreviation for poly-purine (A and/or G). In some embodiments, a poly-(r) sequence comprises (i) a randomized sequence comprising G and A nucleotides and (ii) a non-randomized repeating (A and G) sequence. Also provided are uses of such populations for purifying or separating target nucleic acids from a mixture and methods of purifying or separating target nucleic acids from a mixture using such populations.

The populations of capture probes can bind target nucleic acids without a requirement for a particular sequence in the target and thus can be used to capture a variety of known or unknown target nucleic acids. In some embodiments, the capture probes are attached to a support, e.g., by binding specifically to an immobilized probe on the support. In this way, the capture probes along with target nucleic acid can be separated from other sample components. In some embodiments, capture probe populations contain a first region comprising a non-random or random polymer sequence and a second region comprising a specific binding partner (SBP). The polymer sequence hybridizes nonspecifically to the target nucleic acid and the SBP binds to a second specific binding partner (SBP2), which may be attached to an immobilized probe or to the support. Some embodiments of capture probes include a first region comprising random polymer sequence made up of guanine (G) and adenine (A) nucleotides, which may be deoxyribonucleotides, ribonucleotides, and/or 2′-O-methyl modified RNA residues (also referred to as 2′-O-Me nucleotides). Some embodiments include one or more base analogs (e.g., inosine, 5-nitroindole) or abasic positions in the random polymer sequence. Some embodiments include a random polymer sequence that contains one or more sequences of poly-(r) bases, i.e., a random mixture of G and A bases (e.g., see WIPO Handbook on Industrial Property Information and Documentation, Standard ST.25 (1998), Table 1). G bases were chosen for their “wobble” property, i.e., G binds C or U/T. It is understood that synthesizing capture probes with a random polymer sequence provides a population of oligonucleotides that contain different random polymer sequences made up of the bases included during the synthesis of the random portion. For example, a population of nonspecific capture probes that include a 15 nt random polymer sequence made up of G and A consists of up to 2¹⁵ unique members.

The nonspecific capture probes described herein may exist in many different embodiments. In some embodiments, they may be represented by the structures, RP-SBP or SBP-RP, in which “RP” stands for the randomized or repeating sequence (first region) and “SBP” stands for the “specific binding partner” (second region). In these representational diagrams, the SBP is represented in a linear manner relative to the RP, but those skilled in the art will appreciate that the SBP may be joined at any point to the RP of the capture probe. Thus, unless otherwise specified, the first and second regions do not necessarily have any particular spatial relationship to each other. In embodiments in which the RP is made up of G and A bases, the nonspecific capture probe may be represented by the diagramed structures (r)ₓ-SBP or SBP-(r)ₓ, in which “r” stands for the G and A bases of the RP portion, “x” stands for the length (in nt) of the r sequence, and “SBP” stands for the “specific binding partner.” Although the SBP and (r)ₓ sequences are shown in a linear manner, it will be understood that the SBP may be joined at any point to the capture probe. In some embodiments, the first region comprises an (r)ₓ sequence wherein x is a value ranging from 2 to 30, e.g., about 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, including but not limited to when x is less than about 12, the first region comprises an (r)_{y} sequence wherein the sum x+y is greater than or equal to about 12.

In some embodiments, the first region comprises a non-randomized repeating (A and G) sequence. Specifically, a non-randomized repeating sequence can include direct or inverted repeats, or both. Thus, examples of such a repeating sequence comprising repeats of A and G nucleotides include (AG)(GA)(AG)(GA)(AG)(GA) (SEQ ID NO: 1), (AG)(AG)(AG)(GA)(AG)(AG) (SEQ ID NO: 2), (AAG)(GAA)(GAA)(AAG) (SEQ ID NO: 3), (AAG)(AAG)(AAG)(AAG) (SEQ ID NO: 4), etc., in which the parentheses indicate the constituent repeats but do not have any structural meaning. In some embodiments, the non-randomized repeating sequence comprises one or more partial repeats, e.g.,

(SEQ ID NO: 5)
(AAG)(AAG)(AAG)(AAG)(A).

The first region may consist of a poly-(r) sequence as described herein and optionally a linker as described herein. Alternatively, the first region may consist of a randomized (A and G) sequence as described herein and optionally a linker as described herein. Alternatively, the first region may consist of a non-randomized repeating (A and G) sequence as described herein, a poly-(r) sequence as described herein, and optionally a linker (e.g., a non-nucleotide linker such as a C-9 linker or a nucleotide linker such as an arbitrary sequence, e.g., about 1-10 nucleotides in length).

The SBP component of a nonspecific capture probe may be any member of a specific binding pair that binds specifically to the SBP2 which may be part of an immobilized probe. Some embodiments of specific binding pairs suitable for use as SBP and SBP2 members include receptor and ligand pairs, enzyme and substrate or cofactor pairs, enzyme and coenzyme pairs, antibody (or antibody fragment) and antigen pairs, sugar and lectin pairs, biotin and avidin or streptavidin, ligand and chelating agent pairs, nickel and histidine, and completely or substantially complementary nucleic acid sequences. In some embodiments, the SBP and SBP2 members are substantially complementary nucleic acid sequences, such as complementary homopolymeric sequences, e.g., a capture probe includes a 3′ substantially homopolymeric SBP sequence that hybridizes to a complementary immobilized SBP2 sequence linked to a support. Other embodiments use non-nucleic acid binding pairs, such as biotin that binds specifically with avidin or streptavidin, as the SBP and SBP2 members.

Embodiments of nonspecific capture probes may be synthesized to include any of a variety of nucleic acid conformations, such as standard DNA or RNA oligonucleotides, or oligonucleotides that include one or more modified linkages in which the sugar moieties have substitutions (e.g., 2′ methoxy or 2′ halide), or one or more positions in alternative conformations, e.g., locked nucleic acid (LNA) or protein nucleic acid (PNA) conformation. A capture probe embodiment may include a non-nucleotide compound as a linker (e.g., C-9) that joins random polymer and/or nonrandom repeat segments of the capture probe. Some embodiments of nonspecific capture probes include those in which a random polymer portion is synthesized using 2′-O-methyl modified RNA residues or containing one or more residues in LNA conformation. The choice of conformation(s) to include in oligonucleotide portions of a nonspecific capture probe may depend on the intended target nucleic acid or type of target nucleic acid to be isolated. For example, a nonspecific capture probe synthesized in the random polymer region with 2′-O-methyl modified RNA residues is can be used to capture RNA targets, whereas one synthesized with some LNA conformation in the random polymer region can be used to capture single-stranded DNA (ssDNA) targets. Some embodiments of capture probes include combinations of conformations (e.g., LNA and DNA), which may be adjacent or joined by a linker. In some embodiments, the first region consists of 2′-O-methyl modified RNA residues.

Nonspecific target capture methods are relatively fast and simple to perform, requiring in some embodiments less than an hour to complete, with the target capture reaction requiring in some embodiments as little as 5 minutes of incubation. Optional steps such as washing of the captured nucleic acid to further purify the nucleic acid (e.g., about 20 additional minutes).

In some embodiments, nonspecific target capture involves mixing a sample containing or suspected of containing a target nucleic acid with a nonspecific capture probe, as described herein, in a substantially aqueous solution and conditions that allow the capture probe to hybridize nonspecifically to the target nucleic acid in the mixture. Such conditions may involve elevated temperatures for a short time (e.g., 60° C. for about 15 min) followed by incubation at room temperature (e.g., about 20-25° C. for about 10 to 90 min). Alternatively, the entire incubation may be at room temperature and substantially shorter (e.g., about 5 min). The mixture may also contain an immobilized probe that binds specifically to the nonspecific capture probe via the SBP-SBP2 specific binding pair. The immobilized probe may be introduced into the mixture simultaneously with the capture probe, or before or after the capture probe is mixed with the sample. In some embodiments, the immobilized probe is introduced into the mixture of the sample and the nonspecific capture probe after the capture probe has been incubated with the sample to allow the capture probe and the target nucleic acids to hybridize nonspecifically in solution phase before the capture probe binds with the immobilized probe. In other embodiments, the immobilized probe is introduced into the mixture substantially simultaneously with the capture probe to minimize mixing steps, which is particularly useful for automated systems. In an embodiment that uses a capture probe with a tail sequence as the SBP, the capture probe binds specifically to a complementary sequence (SBP2) that is contained in the immobilized probe under nucleic acid hybridizing conditions to allow the target nucleic acid bound nonspecifically to the capture probe and linked to the support via the immobilized probe to be separated from other sample components.

Following incubation in which the capture hybridizes nonspecifically to the target nucleic acid and binds specifically to the immobilized probe, the complex made up of the immobilized probe, capture probe and target nucleic acid is separated from other sample components by separating the support with the attached complex from the solution phase. Then, optionally washing step(s) may be performed to remove non-nucleic acid sample components that may have adhered to the complex, a component of the complex, or the support. In some embodiments, a washing step is performed in which the complex attached to the support is washed with a substantially aqueous wash solution that maintains the hybridization complex on the support and then the complex attached to the support is separated from the washing solution which contains the other sample components. The captured target nucleic acid may be separated from one or more of the other complex components before subsequent assay steps are performed, or the complex attached to the support may be used directly in a subsequent step(s). Subsequent steps include, e.g., detection of the captured nucleic acid, e.g., using a detection probe, and/or in vitro amplification of one or more sequences contained in the captured nucleic acid.

Although the length of one or more contiguous random sequences contained in a nonspecific capture probe may vary, a poly-(r) sequence of about 12 nt or greater is sufficient for efficient target capture of many targets. The presence of non-random oligonucleotide or non-nucleotide spacers between random poly-(r) sequences in a nonspecific capture probe may affect target capture efficiency. Nonspecific capture probes that include at least part of a random poly-(r) sequence in LNA conformation may be more effective at ssDNA target capture than a nonspecific capture probe of similar length in DNA conformation, and those that contain a mixture of LNA and DNA residues may be more effective than those that contain all poly-(r) sequences in LNA conformation. Nonspecific capture probes that include at least part of a random poly-(r) sequence in LNA conformation may be more effective at target capture of RNA and ssDNA than target capture of double-stranded DNA (dsDNA). Nonspecific capture probes that include at least part of a random poly-(r) sequence in LNA conformation may be more effective at RNA target capture than capture probes in which the same length of random poly-(r) sequence is synthesized by using 2′-methoxy RNA bases. These general parameters may be applied to choose appropriate embodiments of capture probe populations for capture of an intended target nucleic acid or type of target nucleic acid which may be tested by using standard procedures as described in the examples that follow to select a nonspecific capture probe and conditions that provide the desired target capture results.

An immobilized probe may be connected to a support by any linkage that is stable in the hybridization conditions used in the target capture method. Some embodiments use a support of monodisperse particles which can be retrieved from a mixture by using known methods, e.g., centrifugation, filtration, magnetic attraction, or other physical or electrochemical separation. In some embodiments, the monodisperse particles are magnetic microbeads. In some embodiments, magnetic attraction is used to retrieve the particles from the mixture. In some embodiments, the captured target nucleic acid is isolated and concentrated on the support, i.e., target nucleic acid is concentrated on the support compared to its concentration in the initial sample, which may improve sensitivity of subsequent assay steps performed using the captured nucleic acids, such as an amplification assay step.

Target capture probe populations and methods described herein may be used to isolate a plurality (e.g., two or more) of target nucleic acids from the same sample simultaneously because the nonspecific capture probe binds to more than one species of nucleic acid present in a sample. In some embodiments, nonspecific capture probes may be designed and selected for use to preferentially capture a particular type of nucleic acid (e.g. RNA) from a sample that contains a mixture of nucleic acids (e.g., DNA and RNA). In some embodiments, nonspecific capture probes may be selectively removed from a mixture by designing the capture probes to selectively bind to different immobilized probes which are introduced into the mixture and then separated with an attached complex containing the capture probe and the target nucleic acid. For example, a first nonspecific capture probe that binds preferentially to RNA in a DNA and RNA mixture may bind via a first SBP to a first immobilized SBP2 on a first support and a second nonspecific capture probe that binds preferentially to DNA in a DNA and RNA mixture may bind via a second SBP to a second immobilized SBP2 on a second support. Then, by selectively removing the first and second supports with their attached complexes to different regions of an assay system or at different times during an assay, the RNA components of a sample may be selectively separated from DNA components of the same sample.

In an exemplary embodiment, a sample is prepared by mixing the target nucleic acid or solution thereof with a substantially aqueous solution (e.g., a buffered solution containing salts and chelating agent). A portion of the sample is mixed with a reagent that contained in a substantially aqueous solution the nonspecific target capture probe to and an immobilized probe attached to a support (e.g., a magnetic particle) to make a target capture mixture. The target capture mixture is incubated at a suitable temperature to allow formation of a capture complex made up of the nonspecific capture probe, the target nucleic acid, and the immobilized probe attached to the support. The complex on the support is then separated from the solution phase. The complex on the support optionally is washed to remove remaining portions of the solution phase, and the complex on the support was separated from the washing solution. The target nucleic acid associated with the support is detected to provide a qualitative detection or quantitative measurement of the amount of target nucleic acid that was separated from the other sample components. It will be understood that additional oligonucleotides, such as helper oligonucleotides (U.S. Pat. No. 5,030,557, Hogan et al.) and/or amplification primers may be included in a target capture mixture.

Nonspecific target capture probes can be synthesized using in vitro methods (e.g., Caruthers et al., Methods in Enzymology, vol. 154, p. 287 (1987); U.S. Pat. No. 5,252,723, Bhatt; WO 92/07864, Klem et al.). The synthesized oligonucleotides can be made using standard RNA bases and linkages, DNA bases and linkages, RNA bases with 2′ methoxy linkages, DNA bases in LNA conformation, or in oligonucleotides that contain a combination of such structures. Oligonucleotides can be synthesized to include non-nucleotide spacers (e.g., C-9) or nucleic acid analogues (e.g., inosine or 5-nitroindole). In some embodiments, the nonspecific portion(s) of the capture probe typically contain one or a series of positions that are random “r” residues, i.e., G or A bases. In some embodiments, random r residues are synthesized by using a mixture that contains equal amounts of G and A bases. Some embodiments of the nonspecific capture probes include a 5′ portion that contains the first region that hybridizes nonspecifically to a target nucleic acid and a 3′ DNA “capture tail” sequence, e.g., made up of dT₀₋₃dA₁₈₋₃₀ (SEQ ID NO: 9), such as a dT₃dA₃₀ (SEQ ID NO: 10) or dA₃₀ (SEQ ID NO: 11) sequence. The capture tail portion (also sometimes referred to simply as a tail) allows the capture probe (with or without bound target nucleic acid) to become associated with a support attached to poly-dT oligomers and be separated from the solution phase of a target capture mixture. It will be understood that any “tail” sequence or non-nucleic acid specific binding partner (SBP) may be attached to a nonspecific capture probe, and the chosen specific binding partner on the support (SBP2) is a member of a specific binding pair with the SBP.

Embodiments of the nonspecific capture probes described herein use the following nomenclature to abbreviate the structure of the oligonucleotide components in a 5′ to 3′ orientation. An oligonucleotide that contains one or more residues of random G or A bases uses the term “(r)ₓ” where “r” stands for the random assortment of G and A, and “x” designates the number of positions in the random assortment of G and A bases. If the oligomer uses RNA bases with a backbone of 2′-methoxy linkages, the term may also include “2′-Ome” to designate the modified linkages of the random assortment of G and A bases, e.g., 2′-Ome-(r)ₓ. If the oligonucleotide uses standard DNA linkages, the term may include “d” to designate DNA for the random assortment of G and A bases, e.g., d(r)ₓ, whereas if the oligomer uses DNA bases with a locked nucleic acid (LNA) conformation, the term includes “L” to designate the LNA conformation for the random assortment of G and A bases, e.g., L(r)ₓ. An oligonucleotide made up of a combination of different portions may include one or more of these terms to define the entire structure. For example, an oligonucleotide made up of six random G and A bases (r bases) with standard DNA linkages, three T bases with standard DNA linkages, and five random G and A bases (r bases) with standard DNA linkages in a 5′ to 3′ orientation would be abbreviated as d(r)₆-dT₃-d(r)₅ (SEQ ID NO: 12). For another example, an oligonucleotide in a 5′ to 3′ orientation made up of five random G and A bases with LNA linkages, three A bases with DNA linkages, and four random G and A bases with DNA linkages would be abbreviated as L(r)₅-dA₃-d(r)₄ (SEQ ID NO: 13). For another example, an oligonucleotide in a 5′ to 3′ orientation made up of ten random G and A bases with 2′-methoxy linkages and a 3′ tail of thirty A bases with standard DNA linkages would be abbreviated as 2′-Ome-(r)₁₀-dA₃₀ (SEQ ID NO: 14).

In some embodiments, a combination is provided of a population of capture probes as described above and a second population of capture probes comprising a first region that is at least about 12 residues in length and comprises a poly-(k) sequence comprising (i) a randomized sequence comprising G and U/T nucleotides, or (ii) a non-randomized repeating (G and U/T) sequence; and a second region comprising a third specific binding partner (SBP3), wherein the SBP3 is capable of specifically binding a fourth specific binding partner (SBP4). Exemplary second populations are described in Becker et al., US 2013/0209992 (Aug. 15, 2013), which is incorporated herein by reference. Capture probes of the second population may be described using (k)ₓ nomenclature which parallels the (r)ₓ nomenclature discussed above. “G and U/T nucleotides” includes (i) G and U nucleotides, (ii) G and T nucleotides, or (iii) G, U, and T nucleotides. Similarly, the repeats in a non-randomized repeating (G and U/T) sequence may include (i) G and U nucleotides, (ii) G and T nucleotides, or (iii) G, U, and T nucleotides, and sequences such as (GU) and (GT) are considered repeats of each other notwithstanding the presence of a U in the former and a T in the latter. The second population of capture probes may include RNA, DNA, LNA, and/or 2′-O-methyl modified RNA residues. The SBP4 may be any of the embodiments described above with respect to the SBP2 and is not necessarily identical to the SBP2.

In some embodiments, the SBP (of the population comprising a poly-(r) sequence) and the SBP3 of the second population are capable of binding the same SBP2/SBP4, i.e., the same entity can serve as both SBP2 and SBP4. For example, the SBP2/SBP4 may be a poly-T sequence, and the SBP and SBP3 may be, independently, a dA₃₀ (SEQ ID NO: 11) or a dT₃dA₃₀ (SEQ ID NO: 10) sequence. In some embodiments, the SBP and SBP3 are identical to each other.

In some embodiments, a population of capture probes or combination disclosed herein is provided in a reaction mixture or kit that further comprises an SBP2 immobilized on a support. Examples of the SBP2 are discussed above. The reaction mixture or components of the kit may be provided in dry form or in a solution phase. In some embodiments, the solution phase comprises a detergent, such as lithium or sodium lauryl sulfate. In some embodiments, the solution phase comprises a base, such as lithium hydroxide.

Populations, combinations, reaction mixtures, and kits disclosed herein may be used to separate target nucleic acids from various types of samples. In some embodiments, the sample is from an animal source (e.g., human, non-human vertebrate, non-human mammal), an environmental source (e.g., water, plants, soil), a food source (e.g., food products, food preparation areas) or industrial sources (e.g. bioreactors, cell culture wares, pharmaceutical manufacturing wares, biologic reagents, pharmaceutical reagents). Exemplary animal or human sources include peripheral blood, serum, plasma, cerebrospinal fluid, sputum, or a swab specimen (e.g., a nasopharyngeal, buccal, wound, vaginal, or penile swab). Accordingly, in some embodiments, a reaction mixture further comprises a sample such as any of the foregoing. In some embodiments, a target nucleic acid is associated with members of a population of target capture probes in a reaction mixture. The target nucleic acid may be of viral, prokaryotic, eukaryotic, or synthetic origin or a combination thereof, and may be DNA, RNA, modified nucleic acid, or a combination thereof.

Examples are included to describe embodiments of the disclosed nonspecific target capture methods and compositions. Exemplary reagents in target capture procedures described below are as follows, although those skilled in the art of molecular biology will appreciate that many different reagents are available to perform the basic steps of the reactions and tests described. Sample transport reagent: 110 mM lithium lauryl sulfate (LLS), 15 mM NaH₂PO₄, 15 mM Na₂HPO₄, 1 mM EDTA, 1 mM EGTA, pH 6.7. Target capture reagent (TCR): 250 mM HEPES, 1.88 M LiCl, 310 mM LiOH, 100 mM EDTA, pH 6.4, and 250 μg/ml of paramagnetic particles (0.7-1.05μ particles, Sera-Mag™ MG-CM) with (dT)₁₄ oligomers covalently bound thereto. Wash Solution: 10 mM HEPES, 150 mM NaCl, 6.5 mM NaOH, 1 mM EDTA, 0.3% (v/v) ethanol, 0.02% (w/v) methyl paraben, 0.01% (w/v) propyl paraben, and 0.1% (w/v) sodium lauryl sulfate, pH 7.5. Hybridization reagent: 100 mM succinic acid, 2% (w/v) LLS, 100 mM LiOH, 15 mM aldrithiol-2, 1.2 M LiCl, 20 mM EDTA, and 3.0% (v/v) ethanol, pH 4.7. Selection reagent: 600 mM boric acid, 182.5 mM NaOH, 1% (v/v) octoxynol (TRITON® X-100), pH 8.5 or pH 9.2, to hydrolyze labels on unhybridized detection probe oligomers. Detection reagents comprise Detect reagent I: 1 mM nitric acid and 32 mM H₂O₂, and Detect reagent II: 1.5 M NaOH, to produce chemiluminescence from labels (see U.S. Pat. Nos. 5,283,174, 5,656,744, and 5,658,737).

Captured target nucleic acids may be detected by using any process that detects nucleic acids. For example, the captured nucleic acids may to detected by using dyes that bind selectively to nucleic acids in general or selectively to a particular form of nucleic acid. Specific nucleic acids may be detected by binding a detection probe that hybridizes specifically to a target sequence in a captured nucleic acid, or target sequences in the captured nucleic acids may be treated by in vitro nucleic acid amplification to amplify part of the captured nucleic acid which then is detected. In some embodiments, the target nucleic acid in the sample is labeled by hybridizing it to a specific detection probe. Detection probe hybridization can occur before, concurrently with, and/or after target capture. An exemplary form of detection probe is labeled with an acridinium ester (AE) compound that produces a chemiluminescent signal (expressed as relative light units or “RLU”) in a homogeneous system by using well known procedures described in detail elsewhere (U.S. Pat. No. 5,658,737, see column 25, lines 27-46, and Nelson et al., 1996, Biochem. 35:8429-8438 at 8432).

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

### EXAMPLES

The following examples are provided to illustrate certain disclosed embodiments and are not to be construed as limiting the scope of this disclosure in any way.

### Example 1—Recovery of a Short DNA Fragment Using a (r)₁₈ and (k)₁₈/(r)₁₈ Capture Probes

This example demonstrates the use of an (r)₁₈-containing and (k)₁₈-containing target capture probe populations by themselves or in combination with each other to capture a short DNA fragment. The (k)₁₈ and (r)₁₈ capture probes used in this experiment comprised a target hybridizing sequence (randomized (k)₁₈ and (r)₁₈ in which the nucleotide residues contained 2′-methoxyribose) and a capture tail was directly joined to the 3′ end of the target hybridizing sequence (the (k)₁₈ or (r)₁₈ sequence), thereby forming a contiguous nucleic acid sequence as shown below.

(r)18 capture probe sequence:
(SEQ ID NO: 6)
5′-RRRRRRRRRRRRRRRRRRTTTAAAAAAAAAAAAAAAAAAAAAAAA
AAAAAA-3′
(k)18 capture probe sequence:
(SEQ ID NO: 7)
5′-KKKKKKKKKKKKKKKKKKTTTAAAAAAAAAAAAAAAAAAAAAAAA
AAAAAA-3′

The stretch of poly-A nucleotides were included to allow the capture probe to hybridize with magnetic microparticles coated with a stretch of poly-T nucleotides. One end of the capture probes hybridizes with the magnetic microparticles and the other end of the capture probes hybridizes non-specifically with the target nucleic acid. By applying a magnetic field, the microparticles with the associated capture probes and target nucleic acids are separated out of solution.

In this experiment, a 500 bp DNA fragment, also referred to as the Adenovirus gene block, corresponding to a region of Adenovirus 1 Hexon gene was used as the target nucleic acid to measure the ability of the (k)₁₈ capture probe by itself, the (r)₁₈ capture probe by itself, or a (k)₁₈/(r)₁₈ capture probe blend to capture short DNA fragments. The sequence of the Adenovirus gene block was as follows: ATGTGCCTTACCGCCAGAGAACGCGCGAAGATGGCTACCCCTTCGATGATGCCGCA GTGGTCTTACATGCACATCTCGGGCCAGGACGCCTCGGAGTACCTGAGCCCCGGGCT GGTGCAGTTCGCCCGCGCCACCGAGACGTACTTCAGCCTGAATAACAAGTTTAGAA ACCCCACGGTGGCGCCTACGCACGACGTGACCACAGACCGGTCTCAGCGTTTGACG CTGCGGTTTATCCCCGTGGACCGCGAGGATACCGCATACTCGTACAAGGCGCGGTTT ACCCTGGCTGTGGGTGACAACCGTGTGCTTGACATGGCTTCCACATACTTTGACATT CGCGGCGTGCTGGACCGGGGCCCCACTTTTAAGCCCTACTCCGGCACTGCCTACAAC GCTCTAGCCCCCAAAGGCGCTCCCAATTCCTGCGAGTGGGAACAAGAAGAACCAAC TCAGGAAATGGCTGAAGAACTTGAAGATGAGGAGGAGGCAGAGGAGGA (SEQ ID NO: 8). The recovery of the Adenovirus gene block was measured by a real-time PCR assay specific for that region of the Adenovirus genome.

The Adenovirus gene block was introduced at a concentration of about 13,888 copies per mL into a matrix comprising of a pool of Adenovirus negative nasopharyngeal (NP) swab specimens. These NP specimens contained a physiological level of non-Adenovirus background nucleic acid. The Adenovirus gene block-containing NP specimens were treated to denature double stranded DNA. 72 μL of the Adenovirus gene block-containing NP specimens (containing about 1000 copies of the Adenovirus gene block) were incubated in a final reaction volume of 936 μL containing 100 μg of poly-T coated magnetic microparticles and either a) 20 picomoles of (k)₁₈ target capture probe, b) 20 picomoles of (r)₁₈ target capture probe, or c) 10 picomoles of (k)₁₈ target capture probe plus 10 picomoles of (r)₁₈ target capture probe. The magnetic microparticles and bound nucleic acid were separated out of the solution by the application of a magnetic field, allowing the supernatant to be removed from the captured target:capture probe:magnetic microparticle combination. The magnetic microparticles were then resuspended in Wash Solution. The resuspended microparticles were subjected to one more round of separation, supernatant removal, and resuspension in Wash Solution. After separation and removal of the second round of Wash Solution, the microparticles were incubated in 50 of Elution Buffer (5 mM Tris in water with preservatives) which disrupts nucleotide hybridizations. The magnetic particles were separated by the application of a magnetic field and the nucleic acid containing eluate was recovered.

Each nucleic acid containing eluate was assayed for the recovery of the Adenovirus gene block by real-time PCR. As a control, pure Adenovirus gene block that did not undergo target capture was assayed at a copy level representing 100% recovery (“Direct Spike” in Table 1). The copy level of the recovered Adenovirus gene block was inferred from the cycle number at which the real time PCR amplification curve crossed a fixed threshold (CT). Table 1 lists the CT values and estimates the percent recovery of the Adenovirus gene block using the different target capture probes.

TABLE 1
Recovery of Adenovirus gene block

| | CT | Percent Recovery |
|---|---|---|
| Direct Spike (no Target Capture) | 32.5 | 100% |
| (k)₁₈ Capture Probe Only | 34.5 | 25.0% |
| (r)₁₈ Capture Probe Only | 32.6 | 93.3% |
| (k)₁₈/(r)₁₈ Capture Probe Blend | 32.6 | 93.3% |

The experiment indicates that the (r)₁₈ capture probe is able to better capture this short sequence of DNA. Importantly, the addition of the (k)₁₈ capture probe to the (r)₁₈ capture probe did not interfere with the recovery of the Adenovirus gene block, and using 10 pmol of the R18 capture probe (in combination with 10 pmol of the (k)₁₈ capture probe) gave similar results to using 20 pmol of the (r)₁₈ capture probe. The (r)₁₈ and (k)₁₈ target capture probes are compatible when mixed together in a capture reaction.

### Example 2—Recovery of Adenovirus Nucleic Acid from Clinical Specimens Using (k)₁₈/(r)₁₈ Capture Probes

This example demonstrates the increased efficiency of Adenoviral nucleic acid recovery from clinical specimens with the use of (k)₁₈ target capture probe in combination with (r)₁₈ target capture probe ((k)₁₈/(r)₁₈ blend) as compared to the use of (k)₁₈ target capture probe only. The clinical samples used in this study are nasopharyngeal (NP) swab specimens. The (r)₁₈ and (k)₁₈ capture probes used in this experiment were as described above in Example 1.

In this experiment 49 clinical NP specimens, known to be Adenovirus-positive by a comparator assay, were processed using either (k)₁₈/(r)₁₈ blend or (k)₁₈ alone. Briefly, the NP specimens were treated to denature double stranded DNA. The NP specimens were incubated in a final reaction volume of 936 μL containing 100 μg of poly-T coated magnetic microparticles and either a) 20 picomoles of (k)₁₈ target capture probe or b) 10 picomoles of (k)₁₈ target capture probe plus 10 picomoles of (r)₁₈ target capture probe. The magnetic microparticles and bound nucleic acid were separated out of the solution by the application of a magnetic field, allowing the supernatant to be removed from the captured target:capture probe:magnetic microparticle combination. The magnetic microparticles were then resuspended in Wash Solution. The resuspended microparticles were subjected to one more round of separation, supernatant removal, and resuspension in Wash Solution. Upon separation and removal of the second round of Wash Solution, the microparticles were incubated in 50 μL of Elution Buffer which disrupts nucleotide hybridizations. The magnetic particles were separated by the application of a magnetic field and the nucleic acid containing eluate was recovered.

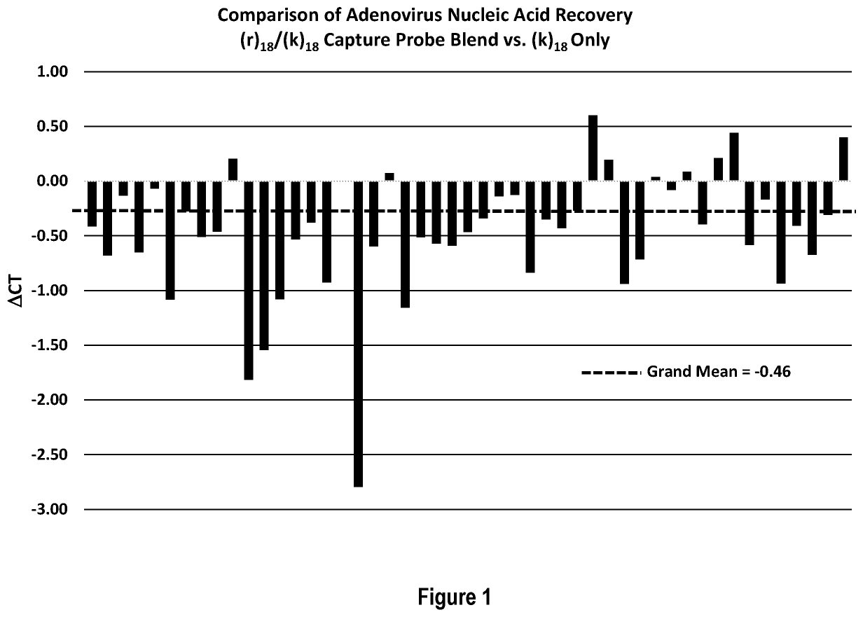

Each nucleic acid containing eluate was assayed for the recovery of Adenovirus nucleic acid by real-time PCR. The relative difference in Adenovirus nucleic acid recovery between the two test conditions was inferred by a comparison of the cycle numbers at which the real-time PCR curves crossed a fixed threshold (CT). The delta CT (ΔCT) is defined as the CT of (k)₁₈/(r)₁₈ blend extraction minus the CT of (k)₁₈ only extraction. A negative ΔCT indicates that the (k)₁₈/(r)₁₈ blend recovered more Adenovirus nucleic acid while a positive ΔCT indicates that the (k)₁₈ only recovered more Adenovirus nucleic acid. FIG. 1 plots the ΔCT of all 49 clinical specimens.

The data indicates that the (k)₁₈/(r)₁₈ blend recovered more Adenovirus nucleic acid in 39 out of 49 specimens. In these specimens, the average ΔCT was −0.64 which represents a 56% increase in Adenovirus nucleic acid recovery using the (k)₁₈/(r)₁₈ blend. Across all 49 specimens, the average ΔCT was −0.46 which represents a 38% increase in Adenovirus DNA recovery using the (k)₁₈/(r)₁₈ blend.

What is claimed is:

1. A population of capture probes for isolating a target nucleic acid from a sample, comprising a first region that is at least about 12 residues in length and comprises at least one poly(r) sequence comprising (i) a randomized sequence comprising G and A nucleotides, or (ii) a non-randomized repeating (A and G) sequence; and a second region comprising a first specific binding partner (SBP), wherein the SBP is capable of specifically binding a second specific binding partner (SBP2).

2. The population of capture probes of claim 1, wherein the poly-(r) sequence comprises the randomized sequence comprising G and A nucleotides.

3. The population of capture probes of claim 2, wherein the first region comprises at least about 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides of randomized poly-(r) sequence.

4. The population of capture probes of any one of the preceding claims, wherein the poly-(r) sequence comprises at least about 4, 5, 6, 7, 8, 9, 10, 11, 12, 13, 14, 15, 16, 17, 18, 19, 20, 21, 22, 23, 24, 25, 26, 27, 28, 29, or 30 nucleotides of a non-randomized repeating (A and G) sequence.

5. The population of capture probes of any one of the preceding claims, wherein the first region is at least 13, 14, 15, 16, 17, 18, 19, 20, 21, 22, 23, 24, 25, 26, 27, 28, 29, or 30 nucleotides in length.

6. The population of capture probes of any one of the preceding claims, wherein the first region consists of the randomized G and A nucleotides, the non-randomized repeating (A and G) sequence, or a combination thereof.

7. The population of capture probes of any one of claims 1-5, wherein the first region further comprises a linker sequence between the poly(r) sequence and a second poly(r) sequence, and the second poly(r) sequence comprises (i) a randomized sequence comprising G and A nucleotides, or (ii) a non-randomized repeating (A and G) sequence.

8. The population of capture probes of claim 7, wherein the poly(r) sequence is at least about 6 residues in length and the second poly(r) sequence is at least about 6 residues in length.

9. The population of capture probes of any one of the preceding claims, wherein the first region comprises 2′-O-methyl modified RNA residues.

10. The population of capture probes of any one of the preceding claims, wherein the first region comprises a poly-(r)₁₈, poly-(r)₂₄, or poly-(r)₂₅ sequence.

11. The population of capture probes of any one of the preceding claims, wherein the SBP is a non-nucleic acid moiety.

12. The population of capture probes of any one of claims 1 to 10, wherein the SBP comprises a homopolymeric sequence.

13. The population of capture probes of claim 12, wherein the SBP comprises a dT₃dA₃₀ (SEQ ID NO: 10) or dA₃₀ (SEQ ID NO: 11) sequence.

14. The population of capture probes of any one of the preceding claims, wherein the SBP is situated 3′ to the first region.

15. A combination comprising the population of capture probes of any one of the preceding claims and a second population of capture probes comprising a first region that is at least about 12 residues in length and comprises a poly-(k) sequence comprising (i) a randomized sequence comprising G and U/T nucleotides, or (ii) a non-randomized repeating (G and U/T) sequence; and a second region comprising a third specific binding partner (SBP3), wherein the SBP3 is capable of specifically binding a fourth specific binding partner (SBP4).

16. The combination of claim 15, wherein the SBP and the SBP3 are capable of binding the same SBP2/SBP4.

17. The combination of claim 16, wherein the SBP and the SBP3 are identical to each other.

18. The combination of any one of claims 15 to 17, wherein the first region of the second population is at least 13, 14, 15, 16, 17, 18, 19, 20, 21, 22, 23, 24, 25, 26, 27, 28, 29, or 30 nucleotides in length.

19. The combination of any one of claims 15 to 18, wherein the first region of the second population comprises a poly-(k)₁₈, poly-(k)₂₄, or poly-(k)₂₅ sequence.

20. The combination of any one of claims 15 to 19, wherein the first region of the second population consists of randomized G and U/T nucleotides or non-randomized repeating (G and U/T).

21. A kit or reaction mixture for isolating a target nucleic acid from a sample, said reaction mixture comprising:
a. a population of capture probes of any of claims 1 to 14 or combination of any one of claims 15 to 20; and
b. an SBP2 immobilized on a support.

22. The kit or reaction mixture of claim 21, wherein the SBP and SBP2 are substantially complementary nucleic acid sequences.

23. The kit or reaction mixture of claim 21, wherein the SBP and SBP2 are non-nucleic acid moieties.

24. The kit or reaction mixture of any one of claims 21-23, further comprising a detergent.

25. The kit or reaction mixture of any one of claims 21-24, further comprising lithium or sodium lauryl sulfate and/or lithium hydroxide.

26. The kit or reaction mixture of any one of claims 21-25, comprising the combination of capture probes of any one of claims 15-20.

27. The kit or reaction mixture of claim 26, wherein the SBP and the SBP3 are capable of binding the SBP2.

28. The kit or reaction mixture of claim 26, further comprising an SBP4 immobilized on a support.

29. The kit or reaction mixture of any one of claims 21-28, further comprising a solution phase.

30. The reaction mixture of claim 29, wherein the reaction mixture comprises a target nucleic acid in the solution phase and/or associated with the capture probes.

31. The reaction mixture of claim 30, wherein the target nucleic acid is derived from cells that have been treated to release intracellular components into the solution phase.

32. The reaction mixture of any one of claims 29-31, wherein the solution phase comprises a sample from an animal, environmental, food, or industrial source.

33. The reaction mixture of any one of claims 29-32, wherein the solution phase comprises a sample comprising peripheral blood, serum, plasma, cerebrospinal fluid, sputum, or a swab specimen.

34. A method for isolating a target nucleic acid from a sample, the method comprising:
a. contacting a population of capture probes of any one of claims 1 to 14 or combination of any one of claims 15 to 20 with a solution containing nucleic acids to form a reaction mixture, wherein the reaction mixture further comprises a support comprising the SBP2;
b. incubating the reaction mixture in conditions that allow hybridization of the first region with the target nucleic acid and that allow for association of the SBP with the SBP2 immobilized to the support, thereby forming a hybridization complex in contact with a solution phase; and
c. separating the support from the solution phase, thereby isolating the target nucleic acid from other components in the sample.

35. A method for isolating a target nucleic acid from a sample, the method comprising:
a. incubating the reaction mixture of any one of claims 21-33 with the sample in conditions that allow hybridization of the first region with the target nucleic acid and that allow for association of the SBP with the SBP2 immobilized to the support, thereby forming a hybridization complex in contact with a solution phase; and
b. separating the support from the solution phase, thereby isolating the target nucleic acid from other components in the sample.

36. The method of claim 34 or 35, wherein the sample contains cells and is treated before the contacting step to release intracellular components into the solution.

37. The method of claim 36, wherein the treatment comprises treating the sample with a solution containing a detergent.

38. The method of any one of claims 34-37, wherein the sample is from an animal, environmental, food, or industrial source.

39. The method of any one of claims 34-38, wherein the sample comprises peripheral blood, serum, plasma, cerebrospinal fluid, sputum, or a swab specimen.

40. The method of any one of claims 34-39, wherein the sample comprises a cellular lysate.

41. The method of any one of claims 34-40, wherein the SBP and SBP2 are non-nucleic acid moieties.

42. The method of any one of claims 34-40, wherein the SBP and SBP2 are substantially complementary nucleic acid sequences.

43. The method of any one of claims 34-42, wherein the combination as recited in any one of claims 15-20 is contacted with the solution containing nucleic acids.

44. The method of claim 43, wherein the SBP and the SBP3 are capable of binding the SBP2.

45. The method of claim 43, wherein the reaction mixture further comprises a support comprising the SBP4.

46. The population, combination, reaction mixture, or method of any one of the preceding claims, wherein the target nucleic acid comprises DNA.

47. The population, combination, reaction mixture, or method of any one of the preceding claims, wherein the target nucleic acid comprises RNA.

48. The population, combination, reaction mixture, or method of any one of the preceding claims, wherein the target nucleic acid comprises viral nucleic acid.

49. The population, combination, reaction mixture, or method of any one of the preceding claims, wherein the target nucleic acid comprises prokaryotic nucleic acid.

50. The population, combination, reaction mixture, or method of any one of the preceding claims, wherein the target nucleic acid comprises eukaryotic nucleic acid.

51. The population, combination, reaction mixture, or method of any one of the preceding claims, wherein the target nucleic acid comprises synthetic nucleic acid.

52. The population, combination, reaction mixture, or method of any one of the preceding claims, wherein the target nucleic acid comprises a combination of DNA, RNA, viral nucleic acid, bacterial nucleic acid, eukaryotic nucleic acid, and/or synthetic nucleic acid.


SEQUENCE LISTING (14 sequences)

SEQ ID NO: 1 - DNA, 12 bp, Artificial Sequence
  Exemplary non-randomized repeating sequence
aggaaggaagga

SEQ ID NO: 2 - DNA, 12 bp, Artificial Sequence
  Exemplary non-randomized repeating sequence
agagaggaagag

SEQ ID NO: 3 - DNA, 12 bp, Artificial Sequence
  Exemplary non-randomized repeating sequence
aaggaagaaaag

SEQ ID NO: 4 - DNA, 12 bp, Artificial Sequence
  Exemplary non-randomized repeating sequence
aagaagaagaag

SEQ ID NO: 5 - DNA, 13 bp, Artificial Sequence
  Exemplary non-randomized repeating sequence
aagaagaagaaga

SEQ ID NO: 6 - DNA, 51 bp, Artificial Sequence
  (r)18 capture probe sequence
rrrrrrrrrrrrrrrrrrtttaaaaaaaaaaaaaaaaaaaaaaaaaaaaaa

SEQ ID NO: 7 - DNA, 51 bp, Artificial Sequence
  (k)18 capture probe sequence
kkkkkkkkkkkkkkkkkktttaaaaaaaaaaaaaaaaaaaaaaaaaaaaaa

SEQ ID NO: 8 - DNA, 500 bp, Artificial Sequence
  500 bp DNA fragment of Adenovirus 1 Hexon
atgtgccttaccgccagagaacgcgcgaagatggctaccccttcgatgatgccgcagtggtcttacatgcacatctcgggccaggacgcctcggagtacctgagccccgggctggtgcagttcgcccgcgccaccgagacgtacttcagcctgaataacaagtttagaaaccccacggtggcgcctacgcacgacgtgaccacagaccggtctcagcgtttgacgctgcggtttatccccgtggaccgcgaggataccgcatactcgtacaaggcgcggtttaccctggctgtgggtgacaaccgtgtgcttgacatggcttccacatactttgacattcgcggcgtgctggaccggggccccacttttaagccctactccggcactgcctacaacgctctagcccccaaaggcgctcccaattcctgcgagtgggaacaagaagaaccaactcaggaaatggctgaagaacttgaagatgaggaggaggcagaggagga

SEQ ID NO: 9 - DNA, 33 bp, Artificial Sequence
  Non-specific capture probe
  misc_feature (1)..(3): t  is present or absent in each position
  misc_feature (19)..(30): a  is present or absent in each position
tttaaaaaaaaaaaaaaaaaaaaaaaaaaaaaa

SEQ ID NO: 10 - DNA, 33 bp, Artificial Sequence; identical to SEQ ID NO: 9
  Non-specific capture probe

SEQ ID NO: 11 - DNA, 30 bp, Artificial Sequence
  Non-specific capture probe
aaaaaaaaaaaaaaaaaaaaaaaaaaaaaa

SEQ ID NO: 12 - DNA, 14 bp, Artificial Sequence
  exemplary oligonucleotide
rrrrrrtttrrrrr

SEQ ID NO: 13 - DNA, 12 bp, Artificial Sequence
  exemplary oligonucleotide
  misc_feature (1)..(5): locked nucleic acid (LNA) conformation
rrrrraaarrrr

SEQ ID NO: 14 - DNA, 40 bp, Artificial Sequence
  exemplary oligonucleotide
  misc_feature (1)..(10): 2′-methoxy linkages
rrrrrrrrrraaaaaaaaaaaaaaaaaaaaaaaaaaaaaa